(19) United States
(12) Patent Application Publication
(10) Pub. No.: US 2022/0221468 A1
(43) Pub. Date: Jul. 14, 2022

(54) SYSTEMIC ALLERGIC RESPONSE RISK ASSESSMENT IN PEANUT ORAL IMMUNOTHERAPY

(71) Applicant: Aimmune Therapeutics, Inc., Brisbane, CA (US)

(72) Inventor: Stephen G. DILLY, San Mateo, CA (US)

(21) Appl. No.: 17/595,193

(22) PCT Filed: Mar. 20, 2020
(86) PCT No.: PCT/US2020/023903

§ 371 Date: Nov. 10, 2021

Related U.S. Application Data
(60) Provisional application No. 62/897,086, filed on Sep. 6, 2019.
(60) Provisional application No. 62/822,705, filed on Mar. 22, 2019.

(51) Int. Cl. G01N 33/68; A61K 39/35; A61K 9/00; A61P 37/08

(52) U.S. Cl. CPC G01N 33/6854; A61K 39/35; G01N 2800/24; A61P 37/08; A61K 9/0053

(57) ABSTRACT

Described herein are methods of assessing risk of a systemic allergic response in a subject being treated for a peanut allergy by an oral immunotherapy. The oral immunotherapy includes administering to the subject a composition comprising peanut protein according to an oral immunotherapy schedule comprising an up-dosing phase and a maintenance phase. The methods can include obtaining a peanut-specific IgE level when the subject tolerates the dose of 1000 mg or more peanut protein; and assessing the risk of a systemic allergenic response in the subject based on the obtained peanut-specific IgE level.

### CROSS-REFERENCE TO RELATED APPLICATIONS

This application claims priority benefit to U.S. Provisional Application No. 62/822,705, filed on Mar. 22, 2019; and U.S. Provisional Application No. 62/897,086, filed on Sep. 6, 2019, each titled “SYSTEMIC ALLERGIC RESPONSE RISK ASSESSMENT IN PEANUT ORAL IMMUNOTHERAPY”; each of which are incorporated herein by reference for all purposes.

### FIELD OF THE INVENTION

Described herein are methods for treating peanut allergy using an oral immunotherapy comprising an up-dosing phase and a maintenance phase.

### BACKGROUND OF THE INVENTION

Peanut allergy is an allergic hypersensitivity reaction of the immune system to peanut protein. Peanut allergy often develops in childhood and is usually a lifelong affliction. Allergic reactions to peanut can be severe and life threatening, and are a major source of severe food-induced anaphylaxis.

Until recently, the standard of care for treating peanut allergy included dietary elimination and avoidance of peanuts, education on the signs of anaphylaxis, and administration of injectable epinephrine in response to severe allergic reactions with dietary exposure to peanut protein. However, accidental ingestion of peanuts is common, due to difficulty in interpreting food labels and the presence of undeclared ingredients in unlabeled food. Oral immunotherapy (OIT) is a promising new treatment for peanut allergy. See, for example, Bird et al., Efficacy and Safety of AR101 in Oral Immunotherapy for Peanut Allergy: Results of ARC001; a randomized Double-Blind, Placebo-Controlled Phase 2 Clinical Trial, J. Allergy Clin. Immunol. Pract., vol. 6, no. 2, p. 476-485 (2018). Peanut OIT includes exposing patients to gradually increasing doses of peanut protein to induce desensitization, which is intended to reduce the risk of a serious reaction upon accidental exposure to peanut.

A recent phase III clinical study, Peanut Allergy Oral Immunotherapy Study of AR101 for Desensitization in Children and Adults (PALISADE), studied the efficacy and safety of AR101, a composition containing peanut protein, administered according to an oral immunotherapy schedule that included an up-dosing phase and a 6 month maintenance phase. Vickery et al., AR101 Oral Immunotherapy for Peanut Allergy, New England Journal of Medicine, vol. 279, pp. 1991-2001 (2018). During the PALISADE study, it was observed that median peanut-specific IgE levels increased following up-dosing, and decreased following the 6 month maintenance phase. Additionally, median peanut-specific IgG4 levels increased following up-dosing, and further increased following the 6 month maintenance phase. It was further determined that 84.5% of patients receiving AR101 and completing treatment tolerated a 600 mg dose of peanut protein.

Although peanut OIT has been shown to be effective at inducing a desensitized state in many subjects, the safety in administering allergenic compositions to allergic patients should be considered to avoid dangerous allergic reactions.

### SUMMARY OF THE INVENTION

Described herein are methods of assessing risk of a systemic allergic response in a subject being treated for a peanut allergy by oral immunotherapy, which includes orally administering a composition comprising peanut protein to the subject according to an oral immunotherapy schedule. The oral immunotherapy schedule comprises an up-dosing phase and a maintenance phase. In some embodiments, the risk of systemic allergic response is assessed in the subject after the subject has become desensitized to peanut protein.

Provided herein is a method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising obtaining a peanut-specific IgE level of the subject during the maintenance phase; and assessing the risk of a systemic allergenic response for the subject based on the obtained peanut-specific IgE level, wherein a peanut-specific IgE level above a predetermined threshold (such as about 70 kU_{A}/L) indicates an increased risk of a systemic allergic response.

Also provided herein is method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising obtaining a peanut-specific IgE level of the subject during the maintenance phase; and assessing the risk of a systemic allergenic response for the subject based on the obtained peanut-specific IgE level, wherein a peanut-specific IgE level above a baseline peanut-specific IgE level of the patient indicates an increased risk of a systemic allergic response compared to a baseline risk.

In some embodiments, the subject tolerates a dose of about 1000 mg or more of peanut protein when the peanut-specific IgE level is obtained and the risk of the systemic allergic response is assessed. In some embodiments, the subject tolerates a cumulative dose of about 2043 mg or more of peanut protein when the peanut-specific IgE level is obtained and the risk of the systemic allergic response is assessed.

In some embodiments, the method further comprises administering to the subject the composition comprising the peanut protein according to the oral immunotherapy schedule.

Also provided herein is a method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising desensitizing the subject to peanut protein using the oral immunotherapy until the subject tolerates a dose of 1000 mg or more peanut protein; obtaining a peanut-specific IgE level when the subject tolerates the dose of 1000 mg or more peanut protein; and assessing the risk of a systemic allergenic response in the subject based on the obtained peanut-specific IgE level, wherein a peanut-specific IgE level above a predetermined threshold (such as about 70 kU_{A}/L) indicates an increased risk of a systemic allergic response.

Also provided is a method of assessing risk of a systemic allergic response in a subject during the course of treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising: obtaining a peanut-specific IgE level of the subject prior to the start of the oral immunotherapy; and assessing the risk of a systemic allergic response for the subject during the course of the oral immunotherapy based on the obtained peanut-specific IgE level, wherein a peanut-specific IgE level above a predetermined threshold (such as about 70 kU_{A}/L) indicates an increased risk of a systemic allergic response during the course of oral immunotherapy. In some embodiments, the method further comprises comprising administering to the subject the composition comprising the peanut protein according to the oral immunotherapy schedule. In some embodiments, the risk of systemic allergic response is assessed prior to the start of the oral immunotherapy.

Further provided is a method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising desensitizing the subject to peanut protein using the oral immunotherapy until the subject tolerates a dose of 1000 mg or more peanut protein; obtaining a peanut-specific IgE level when the subject tolerates the dose of 1000 mg or more peanut protein; and assessing the risk of a systemic allergenic response in the subject based on the obtained peanut-specific IgE level, wherein a peanut-specific IgE level above a baseline peanut-specific IgE level of the patient indicates an increased risk of a systemic allergic response compared to a baseline risk.

In some embodiments of the method of assessing risk of a systemic allergic response, the age the subject is obtained, and assessment of the risk of a systemic allergic response is further based on the age of the subject, wherein an age of 12 years of age or older indicates an increased risk of a systemic allergic response compared to an age between about 4 years old and about 11 years old. In some embodiments, the age the subject is obtained, and assessment of the risk of a systemic allergic response is further based on the age of the subject, wherein an age between 12 years of age and 17 years of age indicates an increased risk of a systemic allergic response compared to an age between about 4 years old and about 11 years old. In some embodiments, the sex the subject is obtained, and assessment of the risk of a systemic allergic response is further based on the sex of the subject, wherein the subject being female indicates an increased risk of a systemic allergic response compared to the subject being male. In some embodiments, the sex and age of the subject are obtained, and assessment of the risk of a systemic allergic response is further based on the sex and age of the subject, wherein the subject being female 12 years of age or older indicates an increased risk of a systemic allergic response compared to the subject not being a female 12 year of age or older. In some embodiments, the sex and age of the subject are obtained, and assessment of the risk of a systemic allergic response is further based on the sex and age of the subject, wherein the subject being female between 12 years of age and 17 years of age indicates an increased risk of a systemic allergic response compared to the subject not being a female between 12 year of age and 17 years of age.

Also provided herein is a method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising: obtaining the age of the subject; and assessing the risk of a systemic allergic response for the subject during the course of the oral immunotherapy based on the age, wherein an age of 12 years of age or older indicates an increased risk of a systemic allergic response compared to an age between about 4 years old and about 11 years old. In some embodiments, an age between 12 years of age and 17 years of age indicates an increased risk of a systemic allergic response compared to an age between about 4 years old and about 11 years old. In some embodiments, the sex the subject is obtained, and assessment of the risk of a systemic allergic response is further based on the sex of the subject, wherein the subject being female indicates an increased risk of a systemic allergic response compared to the subject being male. In some embodiments, the method further comprises obtaining a baseline peanut-specific IgE level, wherein assessment of the risk of a systemic allergic response during the course of the oral immunotherapy is further based on the obtained peanut-specific IgE level, and wherein a peanut-specific IgE level above a predetermined threshold (such as about 70 kU_{A}/L) indicates an increased risk of a systemic allergic response during the course of oral immunotherapy.

Further provided herein is a method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising: obtaining the sex of the subject; and assessing the risk of a systemic allergic response for the subject during the course of the oral immunotherapy based on the sex, wherein the subject being female indicates an increased risk of a systemic allergic response compared to a male subject. In some embodiments, the method further comprises obtaining a baseline peanut-specific IgE level, wherein assessment of the risk of a systemic allergic response during the course of the oral immunotherapy is further based on the obtained peanut-specific IgE level, and wherein a peanut-specific IgE level above a predetermined threshold (such as about 70 kU_{A}/L) indicates an increased risk of a systemic allergic response during the course of oral immunotherapy.

Also described herein is a method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising: obtaining the age and sex of the subject; and assessing the risk of a systemic allergic response for the subject based on the age and the sex, wherein the subject being female 12 years of age or older indicates an increased risk of a systemic allergic response compared to the subject not being a female 12 year of age or older. In some embodiments, the subject being female between 12 years of age and 17 years of age indicates an increased risk of a systemic allergic response compared to the subject not being a female between 12 year of age and 17 years of age. In some embodiments, the method further comprises obtaining a baseline peanut-specific IgE level, wherein assessment of the risk of a systemic allergic response during the course of the oral immunotherapy is further based on the obtained peanut-specific IgE level, and wherein a peanut-specific IgE level above a predetermined threshold (such as about 70 kU_{A}/L) indicates an increased risk of a systemic allergic response during the course of oral immunotherapy.

In some embodiments of the methods described herein, the risk of a systemic allergic response during the course of the oral immunotherapy is assessed prior to the start of the oral immunotherapy.

In some embodiments, obtaining the peanut-specific IgE level comprises measuring the peanut-specific IgE level. In some embodiments, obtaining the peanut-specific IgE level comprises receiving the peanut-specific IgE level.

In some embodiments, the peanut-specific IgE level is obtained from the subject less than 6 months from the start of the maintenance phase. In some embodiments, the peanut-specific IgE level is obtained from the subject about 3 months to less than 6 months from the start of the maintenance phase. In some embodiments, the peanut-specific IgE level is obtained from the subject about 3 months from the start of the maintenance phase.

In some embodiments of the methods described herein, the subject undergoes heightened monitoring for a systemic allergic response if the subject is at an increased risk of a systemic allergic response. In some embodiments, the method further comprises advising the subject to avoid strenuous activity within two hours of administering the composition comprising peanut protein if the subject is determined to be at an increased risk of a systemic allergic response. In some embodiments, the method further comprises providing a therapeutically effective amount of epinephrine to the subject for administration in response to a systemic allergic response if the subject is determined to be at an increased risk of a systemic allergic response.

In some embodiments, the maintenance phase comprises administering to the subject a maintenance phase dose comprising the composition comprising the peanut protein a daily basis. In some embodiments, the maintenance phase comprises administering to the subject a maintenance phase dose comprising about 300 mg or more of the peanut protein.

In some embodiments, the subject is about 4 years old or older. In some embodiments, the subject is about 4 years old to less than 18 years old.

In some embodiments, the up-dosing phase comprises administering to the subject two or more different doses of the composition comprising the peanut protein comprising between about 0.2 mg allergenic food protein and about the dose of the maintenance phase dose. In some embodiments, the up-dosing phase is between about 20 weeks months and about 44 weeks in length.

In some embodiments, the oral immunotherapy further comprises an initial escalation phase.

### BRIEF DESCRIPTION OF THE DRAWINGS

FIG. 1 illustrates the AR101 Trial in Europe Measuring Oral Immunotherapy Success in Peanut Allergic Children (ARTEMIS) treatment protocol, a clinical study examining the treatment of subjects for a peanut allergy by oral immunotherapy that includes an initial escalation phase, an up-dosing phase, and a three-month maintenance phase that includes daily administration of a maintenance dose. A double-blind placebo controlled food challenge (DBPCFC) was conducted before the start of up-dosing and at the end of the maintenance phase to determine the highest tolerated dose and cumulative tolerated dose at each time point.

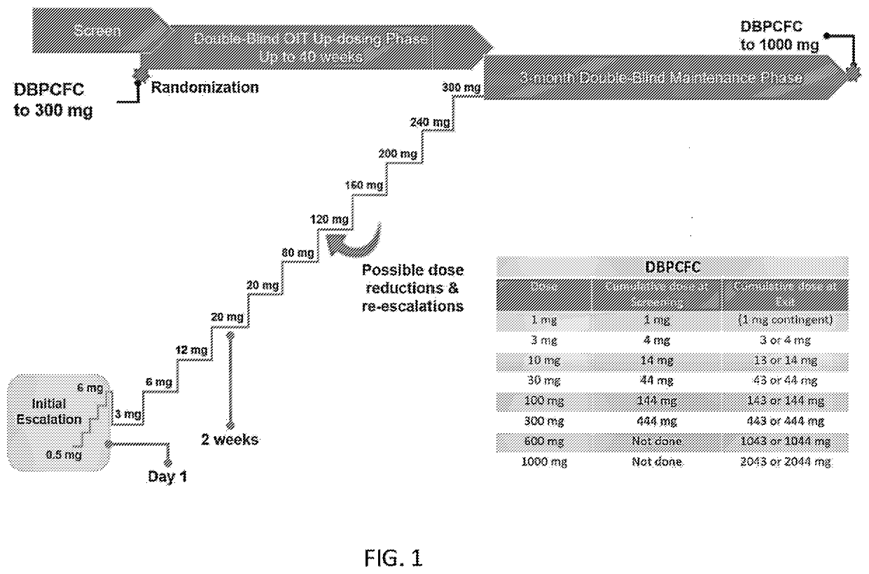

FIG. 2 shows the odds ratio of a patient experiencing a systemic allergic reaction (SAR) by baseline characteristics and demographics in a clinical population. The odds ratios (the ratio of the odds of a SAR occurring under the first condition over the odds of the SAR occurring under the second condition) with a corresponding 95% confidence interval (CI) are reported.

### DETAILED DESCRIPTION OF THE INVENTION

Described herein are methods of assessing risk of a systemic allergic response in a subject being treated food allergy (e.g., a peanut allergy) by oral immunotherapy. Oral immunotherapy generally includes orally administering to the subject a composition comprising allergenic food protein (for example, peanut protein when treating a peanut allergy) according to an oral immunotherapy schedule, which can include an up-dosing phase and a maintenance phase. Risk of a systemic allergic response in the patient can be assessed during the maintenance phase based on a peanut-specific IgE level. An elevated peanut-specific IgE level above baseline (that is, prior to the start of OIT) or above a predetermined threshold (e.g., about 70 kU_{A}/L) indicates an increased risk of systemic allergic response even after desensitization to the peanut protein (for example, after the patient is able to tolerate a 1000 mg dose (and/or 2043 mg cumulative dose), such as assessed by an oral food challenge) of peanut protein. Risk of a systemic allergic response in the patient can also or alternatively be assessed based on one or more demographic factors of the subject, such as the subject's age and/or sex. If the subject has an increased risk of a systemic allergic response, additional precautions can be taken, such as heightened monitoring of the subject, advising the subject to avoid strenuous activity following dose administration and/or providing a therapeutically effective amount of epinephrine to the subject for administration in the event of a systemic allergic response.

It has been discovered that increased tolerance to peanut protein is not entirely associated with a risk of a systemic allergic response due to ingestion of the peanut protein. Patients that become desensitized to the peanut protein as a result of oral immunotherapy can remain at risk for a systemic allergic response. Gastrointestinal symptoms (e.g., nausea, vomiting, etc.) generally decrease after protein desensitization, but the subjects remain at risk for systemic allergic reactions (such as dermatitis, coughing, sneezing, wheezing, etc.) until the level of peanut-specific IgE decreases. As is well-understood in the art, a systemic allergic response is an observed or experienced allergic adverse event with any severity (mild, moderate, or serious, and including anaphylaxis) that involves at least 2 anatomical systems. Non-limiting examples of anatomical systems include the respiratory system, the skin, the gastrointestinal tract, the cardiovascular system, and the nervous system (including, for example, feeling of impending doom, confusion, and anxiety).

Risk of a systemic allergic response in a subject being treated for a peanut allergy by oral immunotherapy can be assessed using one or more factors, such as a peanut-specific IgE level of the patient (such as above or below a baseline peanut-specific IgE level prior to treatment or a pre-determined peanut-specific IgE level threshold, such as about 70 kU_{A}/L), or a predetermined baseline (i.e., prior to the start of treatment) peanut-specific IgE level threshold (such as about 70 kU_{A}/L). It has also been discovered that certain demographics, such as older pediatric patients (e.g., ages 12-17), female patients, or older female pediatric patients (e.g., females ages 12-17), may be at increased risk for systemic allergic responses, even after initial desensitization and some maintenance therapy. Therefore, desensitization to peanut protein is not a complete proxy for completion of the oral immunotherapy maintenance phase, and should not be used to assess the risk of a systemic allergic response.

Oral immunotherapy is a method of inducing desensitization to an allergen in a subject by regular exposure of the subject to increasing doses of the allergen. For peanut allergy, protocols for OIT typically involve an up-dosing phase (also called a build-up phase) and a maintenance phase. Preferably, the OIT further includes an initial escalation phase, although this phase is optional and not required for treatment. The initial escalation phase involves exposure to small doses of peanut protein under clinical supervision to determine the sensitivity of the patient to the peanut protein. This initial escalation phase generally occurs over the course of several (e.g. three or more) hours to two days. These small doses are increased until the subject reaches a goal dose or a highest tolerated dose for the initial escalation phase. The subject then usually begins an up-dosing phase, with regular consultation with a caregiver, usually beginning with the highest tolerated dose administered in the initial escalation phase or a slightly lower dose, and escalating through a series of doses in an up-dosing phase. Additionally, peanut OIT includes a maintenance phase involving continued administration of peanut protein for a period of time. The primary goal of oral immunotherapy is establishing a desensitized state, wherein the subject being treated is less likely to suffer a severe or life-threatening allergic reaction upon accidental exposure to peanut protein.

After initial treatment during the up-dosing phase, the patient is further treated during a maintenance phase. The maintenance phase dose can be administered for a period of time, and preferably on a daily basis. The maintenance phase can extend, for example, for about 3 months or more, such as about 3 months to about 6 months, or about 3 months to less than about 6 months.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

A “cumulative dose” is a sum of doses administered to a subject during the course of an oral food challenge in a single day.

The term “desensitized” is used herein to refer to an increased reaction threshold to a food allergen by a subject as a result of an oral immunotherapy for the food allergen. Desensitization to a food allergen can be tested using methods known in the art, including an oral food challenge. Desensitization may be partial, wherein the subject tolerates an increased amount of the food allergen compared to prior to treatment, but still reacts to higher doses of the food allergen; or the desensitization may be complete, wherein the patient tolerates all tested doses of the food allergen.

The terms “effective,” “efficacy,” or “effectiveness” are used herein to refer to the ability of a therapy to induce immune modulation, such as desensitization, or sustain a desired immune state, such as a desensitized state, unless otherwise indicated.

As used herein, “maintenance phase” refers to a phase of a peanut protein oral immunotherapy that includes administration of peanut protein (i.e., a maintenance dose) to the patient, and occurs after completion of the up-dosing phase.

As used herein, a “mild allergic adverse event” refers to an observed or experienced OIT-treatment-related allergic adverse event associated with transient discomfort, but does not require immediate medical intervention such as hospitalization or epinephrine, and does not substantially interfere with daily activities.

As used herein, a “moderate allergic adverse event” refers to an observed or experienced OIT-treatment-related allergic adverse event that is associated with discomfort of a sufficient degree to interfere with daily activities and that may prompt medical intervention and/or additional observation.

As used herein, “daily” dosing means administering a dose on each consecutive calendar day. The dose may be administered as a single portion on the calendar day, or subdivided into multiple portions administered within the same calendar day.

As used herein, the phrase “serious allergic adverse event” refers to an observed or experienced OIT-treatment-related allergic adverse event leading to anaphylaxis that requires hospitalization and/or administration of epinephrine or other life-saving medical intervention.

The term “subject” or “patient” is used synonymously herein to describe a human of any age.

A subject “tolerates” a dose when the dose is administered to the subject without any moderate or severe allergic adverse event. A subject is considered to tolerate the dose even if a mild allergic adverse event is observed or experienced.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom; delay in progression of the disease; delay in recurrence of the disease; inhibition of the disease; or partially or fully reducing a response or reaction to an allergen.

An “up-dosing phase” refers to a phase of an oral immunotherapy characterized by a series of increasing food allergen doses, beginning with administration of a dose of food allergen lower than the highest dose administered to the patient during the oral immunotherapy, and ending when the highest dose administered to the patient during the oral immunotherapy is achieved.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Peanut Oral Immunotherapy

A subject having a peanut allergy can be treated for the peanut allergy by administering a series of doses of a peanut protein composition, according to a dosing schedule, to the subject during the course of an oral immunotherapy, thereby desensitizing the subject to the peanut allergy. The maintenance phase can extend, for example, for about 3 months or more, such as about 3 months to about 6 months, or about 3 months to less than about 6 months.

The full length of the oral immunotherapy, for example the duration of the up-dosing phase, may vary between subjects depending on the age, health conditions, the nature and type of peanut allergy, concurrent interventions, and/or complicating indications, among others. The therapy is generally multi-phasic, and includes at least an up-dosing phase and a maintenance phase. In some embodiments, the oral immunotherapies may further include an initial escalation phase preceding the up-dosing phase. The doses of the peanut protein composition administered in the up-dosing and maintenance phases can be periodically adjusted or scheduled to increase, decrease, or stay the same. The size of the doses of the peanut protein composition administered in the up-dosing and maintenance phases can be adjusted as necessary based on the judgment of a subject's medical caregiver and/or the needs of the subject.

Methods of diagnosing peanut allergy are known in the art and include immunological assays (such as peanut-specific IgE), skin prick tests, food challenges, and trial elimination diets. For diagnosis of peanut allergy by food challenge, the subject receives increasing doses of peanut protein. An observed allergic reaction to the peanut protein during the food challenge indicates the subject has a peanut allergy and is a candidate for peanut oral immunotherapy. The judgment of whether a subject reacts to a particular dose during the food challenge depends on the test criteria, which can vary. A reaction in a food challenge can be judged by the severity of symptoms (e.g., mild, moderate, or severe) and/or the observability of the symptom (e.g., whether a symptom is subjectively reported by the patient or objectively observed by the medical caregiver).

A subject undergoing peanut OIT as described herein for treatment of a peanut allergy has a known or suspected peanut allergy. In some embodiments, the subject has previously attempted or completed a peanut protein OIT. In some embodiments, the previous peanut protein OIT was ineffective (for example, by failing to induce acceptable desensitization, producing unacceptable allergic adverse reactions, or failing to impart adequate protection from accidental exposure to peanut protein), was terminated by the patient due to discomfort, inconvenience (for example, due to the daily dosing or frequent clinical visits), or necessity (for example, due to reaction to the peanut protein doses and/or due to allergic adverse events during the course of OIT), or was terminated by the patient's medical caregiver (for example, due to allergic adverse reaction to peanut protein doses and/or due to allergic adverse events during the course of OIT).

A subject undergoing peanut OIT as described herein for treatment of a peanut allergy may be treatment naïve, having never undergone a peanut OIT for the treatment of a peanut allergy. A subject being diagnosed for peanut allergy by diagnostic exposure to peanut protein, such as in a food challenge, but with no other history of clinical exposure to peanut protein, is still considered treatment naïve after the diagnostic exposure for the purposes of this application.

The subject receiving the oral immunotherapy treatment for peanut allergy is a human subject. In some embodiments, the subject is about 4 years or older. In some embodiments, the subject is between 4 years and less than 18 years old. In some embodiments, the subject is 18 years or older. In some embodiments, the subject is between 4 years old and 12 years old. In some embodiments, the subject is between 12 years old and 17 years old.

The up-dosing phase precedes the maintenance phase, and includes administration of a series of escalating doses to reach the maximum dose administered to the subject during the course of oral immunotherapy. The length of time of the up-dosing phase can be adapted according the needs of an individual patient, although is generally completed in about 20 to about 40 weeks. For some patients, the up-dosing phase may last as long as 2 years or more. The up-dosing phase may be extended, for example, if a patient experiences allergic adverse events after beginning a higher dose in the dosing series.

The up-dosing phase of a peanut OIT typically involves incrementally increasing the administered peanut protein dose after a period of time (e.g., approximately every 1-4 weeks). A particular dose in the series is repeatedly (e.g., daily) administered to the patient until advancing to the next dose in the series. In some instances, such as when the subject does not tolerate a particular dose in the series or the subject experiences one or more allergic adverse events, the dose is decreased or the dose in the series is repeated for a period of time prior to advancing to the next dose in the series. The rate of up-dosing (e.g., the length of time an individual dose in the series is administered or the size of the dose increment between doses in the series) may be adjusted based on one or more observed allergic adverse events.

Optionally, the oral immunotherapy includes an initial escalation phase before the up-dosing phase, wherein the subject is administered over the course of one or two days a series of escalating doses. The initial escalation phase is distinguished from the up-dosing phase by a lower dose range, shorter intervals between dose escalations, and, typically, closer monitoring by the subject's medical caregiver. For example, a two day initial escalation may comprise a series of doses from about 0.5 mg to about 6 mg peanut protein, such as individual doses of about 0.5 mg, about 1 mg, about 1.5 mg, about 3 mg, and about 6 mg peanut protein. The highest tolerated dose of the initial escalation phase, or a dose lower than the highest tolerated dose in the initial escalation phase, may be the first dose of the up-dosing phase. If a subject does not tolerate at least a certain dose in the initial escalation phase, the subject may be excluded from the oral immunotherapy. For example, if a subject suffers a serious allergic adverse event after administration of the 0.5 mg, 1 mg, or 1.5 mg peanut protein dose, the subject may not be allowed to proceed to the up-dosing phase. The purposes of the initial escalation phase include calibrating the doses of the up-dosing phase (e.g., the initial dose of the up-dosing phase), and ensuring the suitability of the subject for safely proceeding through an up-dosing phase.

Before, during, or after oral immunotherapy (such as before, during or after a maintenance phase of the oral immunotherapy), a peanut-specific IgE (ps-IgE) level can be obtained. The level or ratio can be obtained by measuring the level or ratio, or receiving the level or ratio from another entity (such as a clinical laboratory).

The level of peanut-specific IgE can be measured from a patient serum sample (i.e., to measure a serum level) or from a patient plasma sample (i.e., to measure a plasma level). Whole blood can be drawn from the patient, and the serum or plasm can be isolated from the whole blood using known methods. The level of the ps-IgE can be measured in vitro, for example, using a quantitative immunoassay. Quantitative immunoassays are known in the art, and can include, but are not limited to, an enzyme-linked immunosorbent assay (ELISA); an alkaline phosphatase immunoassay auto-analyzer, such as an IMMULITE® system (Siemens Healthcare Diagnostics, Erlangen, Germany); a radioallergosorbent test (RAST), or a fluoroenzyme immunoassay auto-analyzer, such as the ImmunoCAP® system (Thermo Fisher Scientific/Phadia, Uppsala, Sweden) or UniCAP™ (Phadia AB, Uppsala, Sweden). A fluorescence enzyme immunoassay (FEIA) auto-analyzer (e.g., ImmunoCAP® system) is a preferred technique, although other techniques may be reliably used. For example, another technique may be used as the level of antibody (e.g., IgE) determined by that technique may be normalized to a measurement by a fluorescence enzyme immunoassay auto-analyzer. That is, a level of antibody (e.g., IgE) can be determined by a technique, and can correspond to a level as measured by a fluorescence enzyme immunoassay auto-analyzer.

The level of ps-IgE obtained during the maintenance phase can be compared to a level or ratio obtained during another time point before or during the oral immunotherapy to assess the risk of a systemic allergic response. For example, the level or ratio obtained during the maintenance phase can be compared to a baseline level or ratio. The baseline level or ratio is the level or ratio obtained from the subject prior to the start of the oral immunotherapy. Generally, during oral immunotherapy the ps-IgE level increases during the up-dosing phase compared to the baseline level, and decreases during the maintenance phase. At some point during the maintenance phase, the ps-IgE level decreases to below the baseline level, which indicates a decreased risk of a systemic allergic response compared to the baseline risk (i.e., the risk prior to the start of oral immunotherapy). However, an obtained ps-IgE level that is increased compared to the baseline level indicates an increased risk of a systemic allergic response compared to the baseline risk.

The subject may be at an increased risk of a systemic allergic response even when the subject has obtained increased tolerance to peanut protein. The level of IgG4 antibodies increases during the up-dosing phase of oral immunotherapy, and remains elevated or further elevates during the maintenance phase. The increased IgG4 level is associated win an increase in tolerance of the peanut protein. However, the IgE level in the patient also increases during the up-dosing phase compared to baseline, and this increased IgE level indicates an increased risk of systemic allergic response.

Assessment of the systemic allergic response risk may be based on one or more factors prior to the start of oral immunotherapy or during the course of oral immunotherapy. For example, a baseline peanut-specific IgE level of the subject prior to the start of oral immunotherapy may be obtained, and the risk of a systemic allergic response occurring during the course of the oral immunotherapy may be assessed based on the baseline peanut-specific IgE level, wherein a baseline peanut-specific IgE level above a predetermined threshold (such as about 70 kU_{A}/L) indicates an increased risk of a systemic allergic response occurring during the course of the oral immunotherapy. In another example, a peanut-specific IgE level of the subject during the course of oral immunotherapy (such as during the maintenance phase) is obtained and compared to a baseline peanut-specific IgE level for the patient (i.e., a peanut-specific IgE level for the patient prior to the start of oral immunotherapy), wherein the obtained peanut-specific IgE level of the patient above the baseline peanut-specific IgE level indicates an increased risk of a systemic allergic response compared to a baseline risk (i.e., the risk at prior to the start of oral immunotherapy). In another example, a peanut-specific IgE level of the subject during the course of oral immunotherapy (such as during the maintenance phase) is obtained and compared to a predetermined peanut-specific IgE level threshold (such as about 70 kU_{A}/L), wherein the obtained peanut-specific IgE level of the patient above the predetermined peanut-specific IgE level threshold indicates an increased risk of a systemic allergic response compared to a patient with a peanut-specific IgE level below the threshold. If the patient has an increased risk of a systemic allergic response, the patient may be subjected to heightened monitoring.

In some embodiments, the risk of systemic allergic response is assessed prior to the start of the oral immunotherapy. In some embodiments, the risk of systemic allergic response is assessed during the course of the oral immunotherapy (such as during the maintenance phase). In some embodiments, the risk of systemic allergic response is assessed after desensitization to peanut protein (such as after desensitization to a dose of 1000 mg of peanut protein or more, or after desensitization to a cumulative dose of 2043 mg of peanut protein or more).

In some embodiments, to assess the risk of systemic allergic response, the level of ps-IgE obtained from the patient during the maintenance phase can be compared to a baseline level or to a predetermined threshold. In some embodiments, if the level of ps-IgE is above the predetermined threshold (such as about 70 kU_{A}/L), the subject is considered to be at an increased risk for systemic allergenic response, and optionally undergoes heightened monitoring. In another embodiment, if the level of ps-IgE is above a baseline level (i.e., a level of ps-IgE for the subject prior to treatment), the subject is at an increased risk for a systemic allergic response compared to the baseline risk (i.e., the risk of systemic allergic response prior to treatment).

The risk of systemic allergic response for a subject may also or alternatively be assessed based on the subject's demographics. In some embodiments, the risk of systemic allergic response for a subject may be assessed based on one or more of the subject's age and/or sex. In some embodiments, the risk of systemic allergic response may be assessed based on the subject's age, wherein a subject between 12-years-old and 17-years-old is at increased risk compared to a subject between 4-years-old and 11-years-old. In some embodiments, the risk of systemic allergic response may be assessed based on the subject's sex, wherein a female subject is at increased risk compared to a male subject. In some embodiments, the risk of systemic allergic response may be assessed based on the subject's age and sex, wherein a female subject between 12-years-old and 17-years-old is at increased risk compared to a male subject between 4-years-old and 11-years-old.

The risk of systemic allergic response may be assessed in subjects that have already become desensitized to peanut protein, as these subject can remain at risk, for example due to the ps-IgE level in the subject. By way of example, if the peanut-specific IgE level of the patient is above a baseline level or above a predetermined threshold (such as about 70 kU_{A}/L), the patient may remain at risk of a systemic allergic response even though the patient has been desensitized to peanut protein. The risk of systemic allergic response may also or alternatively be assessed in subjects that have already become desensitized but remain at risk, for example due to the subject's age and/or sex. Desensitization is generally measured using a food challenge, wherein increasing amounts of peanut protein are administered to the subject to determine the highest dose tolerated by the patient. Different doses are spaced by a period of time, generally about 15 to about 30 minutes, and the subject is observed for an allergenic response during this time period. Dose tolerance can then be assessed according to the highest dose tolerated by the subject, or the highest cumulative dose tolerated by the subject. In some embodiments, the subject tolerates a dose of about 10 mg peanut protein or more, about 30 mg peanut protein or more, about 100 mg peanut protein or more, about 300 mg peanut protein or more, about 600 mg peanut protein or more, or about 1000 mg peanut protein or more when the risk of systemic allergic response is assessed. In some embodiments, the subject tolerates a cumulative dose of about 13 mg peanut protein or more, about 43 mg peanut protein or more, about 443 mg peanut protein or more, about 1043 mg peanut protein or more, or about 2043 mg peanut protein or more.

Risk may be further increased if one or more compounding factors are present in the patient. Such a compounding factor can include, for example, inflammation (which may be systemic inflammation or localized inflammation, for example due to a localized injury or surgery), an infection (for example, a viral, fungal, parasitic, or bacterial infection), an allergic reaction (which may be due to peanut or other allergy causing agent), geographic origin (such as European or North American), clinical history (such as history of atopic dermatitis, allergic rhinitis, anaphylaxis, asthma, and/or multiple food allergy), race group, skin prick test (wheal diameter), or a menstruation period (including whether the subject has experienced menarche).

If the subject is determined to be at an increased risk of a systemic allergic response, precautions can be taken. For example, the subject may undergo heightened monitoring for a systemic allergic response, be advised to limit physical activity for a period of time (e.g., about 2 hours) after a composition comprising peanut protein is administered to the subject, and/or the subject may be provided with a therapeutically effective amount of epinephrine for administration in response to a systemic allergic response.

### Maintenance Phase Dosing Schedules

The maintenance phase of the peanut oral immunotherapy begins after the highest dose of the up-dosing phase is achieved. The maintenance phase can extend, for example, for about 3 months or more, such as about 3 months to about 6 months, or about 3 months to less than about 6 months. In some embodiments, the maintenance phase dose is administered to the subject daily during the maintenance phase. The dosage of peanut protein administered to the subject during the maintenance phase is between about 200 mg and about 1,000 mg peanut protein. For example, in some embodiments, a dose during the maintenance phase is between about 200 mg and about 300 mg peanut protein, about 300 mg and about 500 mg peanut protein, about 500 mg and about 1,000 mg peanut protein, or values and ranges therebetween. In an exemplary embodiment, a maintenance phase dose administered to the subject during the maintenance phase is about 300 mg peanut protein.

### Up-Dosing Phase

The up-dosing phase of an oral immunotherapy comprises administering to the patient a series of escalating doses, beginning with a lower dose than the highest dose of the oral immunotherapy and ending with the highest dose of the oral immunotherapy. Each dose in the series of doses is administered periodically, such as daily. Each dose in the series can comprise daily administration of the peanut protein composition for a period of time, such as about 1 week to about 4 weeks, such as about 2 weeks. After the completion of a particular dose in the series for a period of time, treatment can be advanced to a higher dose in the series. In some embodiments, the up-dosing phase of the treatment comprises a series of between 2 and 10 different dose levels. If a subject tolerates a particular dose level during the up-dosing phase for a period of time, the subject can advance to the next dose level in the series of the up-dosing phase. If a subject does not tolerate a particular dose level during the up-dosing phase for a period of time, the subject may repeat the current dose level in the series. Alternatively, if a subject does not tolerate a particular dose level during the up-dosing phase for a period of time, the subject may return to an earlier dose level in the series. The duration of the up-dosing phase therefore depends on the specific responses of the subject. The subject may repeat doses in the series as many times as necessary to achieve the highest dose in the series. The up-dosing phase ends when the highest dose is tolerated for two weeks.

The pharmaceutical composition of peanut protein of a dose administered during the up-dosing phase comprises between about 0.5 mg and about 5,000 mg of peanut protein, such as about 0.5 mg to about 10 mg peanut protein, about 10 mg to about 100 mg peanut protein, about 100 mg to about 300 mg peanut protein, about 300 mg to about 500 mg peanut protein, about 500 mg to about 1,000 mg peanut protein, about 1,000 mg to about 2,000 mg peanut protein, or about 2,000 mg to about 5,000 mg peanut protein and values and ranges therebetween. In a non-limiting exemplary embodiment, the doses of the up-dosing phase are daily administrations of the maximum tolerated dose of the initial escalation phase, such as 3 mg or 6 mg peanut protein, followed by a series of doses of about 12 mg peanut protein, about 20 mg peanut protein, about 40 mg peanut protein, about 80 mg peanut protein, about 120 mg peanut protein, about 160 mg peanut protein, about 200 mg peanut protein, about 240 mg peanut protein, and about 300 mg peanut protein, wherein each dosage level is administered for about 1 week to about 4 weeks (such as about 2 weeks) before advancing to the next dose in the series. In another exemplary embodiment, the doses of the up-dosing phase are daily administrations of the maximum tolerated dose of the initial escalation phase, such as 3 mg or 6 mg peanut protein, followed by a series of escalating daily doses prescribed by a subject's medical caregiver, wherein each daily dose comprises one or more capsules or sachets selected from the group consisting of: 0.5 mg peanut protein capsules, 1 mg peanut protein capsules, 10 mg peanut protein capsules, 20 mg peanut protein capsules, 100 mg peanut protein capsules, or 300 mg peanut protein sachets, wherein each dosage level is administered for about 1 week to about 4 weeks (such as about 2 weeks) before advancing to the next dose in the series.

The series of doses of the up-dosing phase are distinguished by adjustment of the administered dose. The size of the dose in the series of doses of the up-dosing phase are adjusted periodically, such as between once every week and once every six weeks. In some embodiments, the up-dosing phase comprises weekly dose adjustment, dose adjustment every two weeks, dose adjustment every third week, dose adjust every fourth week, dose adjustment every fifth week, dose adjustment every sixth week, or adjustment as needed based on the judgment of the subject's medical caregiver. The dose may be increased to the next scheduled dose in the series, lowered to a previous in the series in response to an allergic adverse event, maintained for an additional interval at the current dose in the series, increased to a higher dose in the series based on the judgment of the subject's medical caregiver, or decreased to a lower dose in the series based on the judgment of the subject's medical caregiver. In some embodiments, the up-dosing phase is adjusted at any time based on the judgment of the subject's medical caregiver that the subject did not tolerate the current dose in the series.

The up-dosing phase proceeds until the subject achieves the final dose in the up-dosing series. In some embodiments, the up-dosing phase is about 1 month to about 6 months, such as about 1 month to about 3 months, or about 3 months to about 6 months. In some embodiments, the up-dosing phase is about 6 months to about 2 years, such as about 6 months to about 1 year, about 1 year to about 18 months, or about 18 months to about 2 years. In a non-limiting exemplary embodiment, the up-dosing phase continues for 22 weeks to 2 years, depending on the number of dose reductions and re-escalations and dose level repeats, through doses of 12 mg peanut protein, 20 mg peanut protein, 40 mg peanut protein, 80 mg peanut protein, 120 mg peanut protein, 160 mg peanut protein, 200 mg peanut protein, 240 mg peanut protein, and terminating at 300 mg peanut protein. In any of the described embodiments, the up-dosing phase terminates when the subject tolerates the scheduled dose of the final dose in the series of the up-dosing phase for 2 weeks, thereby beginning the maintenance phase.

Each dose of the series of the up-dosing phase may be scheduled to last about 1 week, about 2 weeks, about 3 weeks, about 4 weeks, or values and ranges therebetween. Based on the observation of an allergic adverse event, a subject's caregiver may repeat the subject's current dose in up-dosing series. A particular portion with a particular dose may be repeated as many times as necessary, such as once, two times, three times, or four times, or more, to adequately desensitize a subject to that dose, such as when the subject no longer experiences a moderate or serious allergic adverse event upon accidental (or deliberate) exposure to the food allergen.

### Initial Escalation Phase

Optionally, the oral immunotherapy includes an initial escalation phase preceding the up-dosing phase. The initial escalation phase can ensure the safety and suitability of oral immunotherapy for a particular subject. The initial escalation phase is administered over a short period, such as one or two days, at an appropriate medical facility, such as a doctor's office or allergy clinic. The subject is usually closely monitored by a medical caregiver, who can provide interventions such as epinephrine, albuterol, and diphenhydramine in the event of an allergic adverse reaction that necessitates intervention. The initial escalation phase of the oral immunotherapy, if present, includes administration of a plurality of small doses of the peanut protein composition to the subject. The small doses can be spaced by a period of time, such as about 10 minutes to about 60 minutes, and can include 1, 2, 3, 4, or 5 or more doses.

The initial escalation phase may comprise doses between about 0.5 mg and about 6 mg peanut protein, such as about 0.5 mg to about 1.5 mg peanut protein, about 1.5 mg to about 3 mg peanut protein, or about 3 mg to about 6 mg peanut protein. In a non-limiting example, the initial escalation phase comprises an incremental escalation over one day from about 0.5 mg peanut protein to a maximum of about 6 mg peanut protein in a single day, with single doses of about 0.5 mg, about 1 mg, about 1.5 mg, about 3 mg, and about 6 mg of peanut protein, wherein tolerance of the 3 mg or 6 mg peanut protein dose indicates the subject can safely proceed to an up-dosing phase of an oral immunotherapy.

### Compositions for Oral Immunotherapy

Exemplary compositions for treating peanut allergy are described in detail in U.S. Publication No. 2014/0271721, the contents of which are incorporated by reference herein in its entirety. Exemplary methods for preparing peanut protein formulations are described in detail in U.S. Publication No. 2014/0271836, the contents of which are incorporated by reference herein in its entirety.

A subject having a peanut allergy can be treated for the peanut allergy by administering a series of doses of a peanut protein composition to the subject during the course of a peanut protein oral immunotherapy. The peanut protein composition is preferably a pharmaceutical composition comprising one or more peanut allergen proteins for treating peanut allergy. In some embodiments, peanut proteins may be isolated from peanut flour and, optionally, further comprise one or more diluents, one or more glidants, and one or more lubricants. In some embodiments, the pharmaceutical composition of peanut protein comprises between about 0.05% to about 100% w/w of peanut protein.

In some embodiments, the pharmaceutical composition of peanut protein comprises characterized peanut protein. In some embodiments the characterized peanut protein comprises characterized peanut allergen proteins Ara h1, Ara h2, and/or Ara h6. In one embodiment, a final formulation for treating peanut allergy comprises peanut flour, comprising characterized peanut allergen proteins Ara h1, Ara h2, and/or Ara h6, formulated with a diluent, a glidant, and a lubricant in graduated doses comprising capsules containing between about 0.5 and about 5,000 mg of peanut protein for administration in up-dosing, maintenance, and/or initial escalation phases of an oral immunotherapy.

In any of the methods described herein, the pharmaceutical composition of peanut protein for administration in a maintenance phase of an oral immunotherapy may comprise a dose of between about 200 mg to about 1,000 mg peanut protein, such as between about 200 mg and about 250 mg peanut protein, about 250 mg and about 300 mg peanut protein, about 300 mg and about 500 mg peanut protein, and about 500 mg and about 1,000 mg peanut protein. In a non-limiting preferred embodiment, the dose of peanut protein for administration in the maintenance phase of an oral immunotherapy is about 300 mg peanut protein.

In some embodiments, the pharmaceutical composition of peanut protein for administration in an up-dosing phase of an oral immunotherapy comprises between about 0.5 mg and about 5,000 mg peanut protein, such as individual doses in a series of about 3 mg, about 6 mg, about 10 mg, about 12 mg, about 20 mg, about 40 mg, about 80 mg, about 100 mg, about 120 mg, about 160 mg, about 200 mg, about 240 mg, and about 300 mg peanut protein. In a non-limiting exemplary embodiment, the doses of peanut protein for administration in an up-dosing phase of an oral immunotherapy are daily administration of the maximum tolerated dose of the initial escalation phase, such as about 3 mg peanut protein or about 6 mg peanut protein, followed by a series of escalating daily doses prescribed by a subject's medical caregiver, wherein each daily dose comprises one or more capsules or sachets selected from the group consisting of: about 0.5 mg peanut protein capsules, about 1 mg peanut protein capsules, about 10 mg peanut protein capsules, about 20 mg peanut protein capsules, about 100 mg peanut protein capsules, or about 300 mg peanut protein sachets, wherein each dosage level is administered for about 1 week to about 4 weeks (such as about 2 weeks) before advancing to the next dose.

In the methods described herein, an oral immunotherapy may optionally comprise an initial escalation phase. In some embodiments, the pharmaceutical composition of peanut protein for administration in an initial escalation phase of an oral immunotherapy comprises between about 0.5 and about 6 mg of peanut protein, such as individual doses of about 0.5 mg, about 1 mg, about 1.5 mg, about 3 mg, and about 6 mg peanut protein. In some embodiments, the pharmaceutical composition of peanut protein for administration in an initial escalation phase of an oral immunotherapy comprises between about 0.5 and about 6 mg of peanut protein, such as individual doses of about 0.5 mg, about 1 mg, about 1.5 mg, about 3 mg, about 6 mg, and about 12 mg peanut protein.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1

### Phase 3 Clinical Trial in Europe Measuring Oral Immunotherapy Success of AR101 in Peanut Allergic Children

The following study was a European, multicenter, randomized, double-blind, placebo-controlled clinical trial of the efficacy and safety of a peanut protein formulation (AR101) in a characterized desensitization oral immunotherapy regimen in peanut-allergic individuals, entitled “AR101 Trial in Europe Measuring Oral Immunotherapy Success in Peanut Allergic Children (ARTEMIS).” Child and adolescent patients aged 4-17 years were considered eligible. All participants had a clinical history of peanut allergy, confirmed by a screening double-blind, placebo-controlled food challenge (DBPCFC), and either serum peanut-specific IgE (psIgE)≥0.35 kU_{A}/L by UniCAP™ (Phadia AB, Uppsala, Sweden) within past 12 months and/or peanut skin prick test mean wheal diameter≥3 mm larger than the negative control (e.g., saline) at screening. All eligible subjects experienced dose-limiting symptoms at or before the 300 mg (444 mg cumulative) challenge dose of peanut protein (measured as 600 mg of peanut flour) on the screening DBPCFC conducted in accordance with PRACTALL guidelines.

Key exclusion criteria included history of hemodynamically significant cardiovascular disease; severe or life-threatening episode of anaphylaxis or anaphylactic shock within 60 days of screening DBPCFC; history of eosinophilic esophagitis, other eosinophilic GI disease, chronic, recurrent, or severe gastroesophageal reflux disease, symptoms of dysphagia, or recurrent GI symptoms of undiagnosed etiology; and severe asthma. Patients with a history of severe/life-threatening anaphylaxis were permitted, if the episode occurred≥60 days before screening. Patients living at the same address were excluded from the trial to minimize the chances of inadvertent unblinding or errant administration of the incorrect investigational product. At the end-of-study visit, the exit DBPCFC was to be independently assessed by a physician at the site experienced in the procedure who had not substantially participated in the care of that participant throughout the trial.

Efficacy endpoints: The primary clinical efficacy endpoint was the proportion of subjects who tolerate at least 1,000 mg as a single dose (2,043 mg cumulative) of peanut protein with no more than mild symptoms at the exit DBPCFC. The key secondary endpoints included the proportion of subjects who tolerated at least 600 mg as a single dose (1,043 mg cumulative) of peanut protein with no more than mild symptoms at the exit DBPCFC; the proportion of subjects who tolerated at least 300 mg (443 mg cumulative) of peanut protein with no more than mild symptoms at the exit DBPCFC; and the maximum severity of symptoms occurring following ingestion of peanut protein during the exit DBPCFC. Other secondary efficacy endpoints included the maximum tolerated dose (MTD) with no more than mild symptoms at the exit DBPCFC; the change from baseline in MTD of peanut protein at exit DBPCFC; the use of epinephrine as a rescue medication at exit DBPCFC and comparison to its use at screening DBPCFC; the changes in serum peanut-specific IgE, total IgE, and peanut-specific IgG4 levels; and the change in peanut skin prick test (SPT) wheal diameter.

Initial enrollment: Individuals were screened by double-blind, placebo-controlled food challenge (DBPCFC) for allergy to peanut. The DBPCFC was an abbreviated version of the DBPCFC described in the PRACTALL guidelines, progressing up to a top challenge dose of 300 mg (444 mg cumulative). Individuals who were intolerant of 300 mg or less of peanut protein were enrolled in the study. Individuals who tolerated the 300 mg dose of peanut protein were excluded. 175 enrolled individuals were divided 3:1 into peanut protein oral immunotherapy (OIT) with AR101 and placebo arms. 132 individuals were designated to receive peanut protein OIT and 43 individuals to receive placebo. Characteristics including the age, sex, total IgE, peanut-specific IgE (ps-IgE), peanut-specific IgG4 (ps-IgG4), ratio of ps-IgE/IgG4, mean wheal diameter (calculated as the average of the long and short axis from the peanut wheal minus the average of the long and short axis from the saline wheal), and maximum tolerated dose (MTD) of peanut protein at screening DBPCFC of the study population are summarized in Table 1.

TABLE 1
Study Population Baseline Characteristics

| | AR101 | | Placebo | | Total | |
|---|---|---|---|---|---|---|
| | (N = 132) | | (N = 43) | | (N = 175) | |
| Age (at consent) | | | | | | |
| Mean (SD) | 9.0 | (3.70) | 9.5 | (3.86) | 9.1 | (3.74) |
| Median | 8.0 | | 10.0 | | 8.0 | |
| Q1, Q3 | 6.0, 12.0 | | 6.0, 13.0 | | 6.0, 12.0 | |
| Min, Max | 4, 17 | | 4, 17 | | 4, 17 | |
| Age category | | | | | | |
| 4-11 years | 97 | (73.5%) | 30 | (69.8%) | 127 | (72.6%) |
| 12-17 years | 35 | (26.5%) | 13 | (30.2%) | 48 | (27.4%) |
| Sex | | | | | | |
| Male | 68 | (51.5%) | 27 | (62.8%) | 95 | (54.3%) |
| Female | 64 | (48.5%) | 16 | (37.2%) | 80 | (45.7%) |
| Total IgE (IU/ml) | | | | | | |
| n | 126 | | 43 | | 169 | |
| Mean (SD) | 898.18 | (1202) | 666.70 | (612.30) | 839.28 | (1085.72) |
| Median | 487.50 | | 414.00 | | 464.00 | |
| Q1, Q3 | 231.00, 1080.00 | | 225.00, 872.00 | | 231.00, 1006.00 | |
| Min, Max | 11.0, 7674.0 | | 59.0, 2500.0 | | 11.0, 7674.0 | |
| ps-IgE (kU_{A}/L) | | | | | | |
| n | 126 | | 43 | | 169 | |
| Mean (SD) | 136.49 | (241.99) | 114.03 | (140.68) | 130.77 | (220.49) |
| Median | 43.50 | | 69.70 | | 53.10 | |
| Q1, Q3 | 5.20, 147.00 | | 20.70, 103.00 | | 6.72, 138.00 | |
| Min, Max | 0.4, 1398.0 | | 0.4, 494.0 | | 0.4, 1398.0 | |
| ps-IgG₄ (mgA/L) | | | | | | |
| n | 124 | | 42 | | 166 | |
| Mean (SD) | 0.6 | (1.19) | 0.8 | (1.27) | 0.7 | (1.21) |
| Median | 0.3 | | 0.4 | | 0.3 | |
| Q1, Q3 | 0.1, 0.6 | | 0.2, 1.0 | | 0.1, 0.7 | |
| Min, Max | 0, 11 | | 0, 7 | | 0, 11 | |
| ps-IgE/IgG4 ratio | | | | | | |
| n | 124 | | 42 | | 166 | |
| Mean (SD) | 322.76 | (517.11) | 330.24 | (457.52) | 324.65 | (501.36) |
| Median | 138.55 | | 146.43 | | 138.55 | |
| Q1, Q3 | 30.27, 429.32 | | 47.59, 434.78 | | 32.78, 430.00 | |
| Min, Max | 0.1, 3628.6 | | 0.4, 2245.5 | | 0.1, 3628.6 | |
| Mean wheal diameter (mm) | | | | | | |
| n | 128 | | 42 | | 170 | |
| Mean (SD) | 10.26 | (3.78) | 10.80 | (4.26) | 10.39 | (3.90) |
| Median | 9.50 | | 9.75 | | 9.50 | |
| Q1, Q3 | 7.50, 12.25 | | 8.00, 12.50 | | 8.00, 12.50 | |
| Min, Max | 2.0, 25.0 | | 3.0, 22.5 | | 2.0, 25.0 | |
| Maximum tolerated dose of | | | | | | |
| peanut protein at screening | | | | | | |
| DBPCFC | | | | | | |
| None | 16 | (12.1%) | 3 | (7.0%) | 19 | (10.9%) |
| 1 mg | 13 | (9.8%) | 6 | (14.0%) | 19 | (10.9%) |
| 3 mg | 15 | (11.4%) | 6 | (14.0%) | 21 | (12.0%) |
| 10 mg | 29 | (22.0%) | 11 | (25.6%) | 40 | (22.9%) |
| 30 mg | 28 | (21.2%) | 11 | (25.6%) | 39 | (22.3%) |
| 100 mg | 30 | (22.7%) | 6 | (14.0%) | 36 | (20.6%) |
| 300 mg | 1 | (0.8%) | 0 | (0.0%) | 1 | (0.6%) |

Initial escalation (2 consecutive days): After randomization of eligible subjects, peanut OIT subjects and placebo subjects started at a dose of 0.5 mg of AR101 or placebo, and then received incrementally increasing doses at 20 to 30 minute intervals over the course of a single day to a maximum dose of 6 mg. Subjects who failed to tolerate at least a 3 mg dose were to be considered escalation failures. Subjects who tolerated both the 3 mg and 6 mg doses of study product, or who tolerated the 3 mg, but not the 6 mg dose, underwent confirmatory testing of the tolerability of a 3 mg dose the following day (see Table 2 below for Day-1 Initial Escalation Schedule). Subjects who did not tolerate the confirmatory 3 mg dose were discontinued.

TABLE 2
Initial Escalation Period, Day-1, Dosing Schedule

| | | Cumulative |
|---|---|---|
| | Study Product Dose | Study Product Dose |
| Day-1 | (mg peanut protein | (mg peanut protein |
| Dose # | or placebo) | or placebo) |
| 1 | 0.5 | 0.5 |
| 2 | 1 | 1.5 |
| 3 | 1.5 | 3 |
| 4 | 3 | 6 |
| 5 | 6 | 12 |

Up-dosing: Subjects received daily oral dosing of peanut or placebo OIT for about 20 weeks, if up-dosing proceeded without holding at, or reducing, a dose level; up to 40 weeks, maximum. The up-dosing phase consisted of biweekly progression through dose levels of 3, 6, 12, 20, 40, 80, 120, 160, 200, and 240 mg peanut or placebo. All escalation doses (see up-dosing schedule in Table 3, below) were administered in a clinical research center or other monitored setting. Daily doses were self-administered by the individuals or their caregiver at home. A subject receiving and tolerating the 300 mg dose escalation began the maintenance phase period of the study.

TABLE 3
Up-dosing Period Dosing Schedule

| | Dose | | |
|---|---|---|---|
| Up-dosing | (mg peanut protein | Interval | |
| Dose # | or placebo) | (weeks) | % Increase |
| 1 | 3 | 2 | n/a |
| 2 | 6 | 2 | 100% |
| 3 | 12 | 2 | 100% |
| 4 | 20 | 2 | 67% |
| 5 | 40 | 2 | 100% |
| 6 | 80 | 2 | 100% |
| 7 | 120 | 2 | 50% |
| 8 | 160 | 2 | 33% |
| 9 | 200 | 2 | 25% |
| 10 | 240 | 2 | 20% |
| 11 | 300 | Enter Maintenance | 25% |
| | | Period | |

Maintenance: The subjects who reached the target maintenance dose of 300 mg/day of AR101 or placebo entered an approximately 12-week maintenance period of continued dosing at 300 mg/day, which was extended an additional 4 weeks (for a maximum period maintenance period duration of 16 weeks), or to a total treatment phase duration (initial escalation, up-dosing period, and maintenance period) of 56 weeks, whichever came first. After the initial maintenance phase dose, subjects self-administered the daily 300 mg doses of AR101 or placebo at home.

Exit DBPCFC: Following completion of the approximately 12-week maintenance period peanut OIT and placebo subjects underwent an exit DBPCFC to a maximum 1,000 mg dose (cumulative maximum of 2,043 mg) of peanut protein.

### Results

General intent-to-treat efficacy: The intent-to-treat group includes all individuals enrolled in either the OIT or placebo arms of the study, regardless of treatment adherence, withdrawal from the study, or deviation from the study design. From baseline to exit, peanut-specific IgG4 (psIgG4) increased while SPT wheal diameter and psIgE/psIgG4 decreased in AR101-treated subjects compared with placebo-treated subjects. In the intent-to-treat group, psIgE levels increased to the end of dose escalation, and decreased back to baseline levels during maintenance, with no observed change between treatment groups (AR101 and placebo) from baseline to exit. Subjects receiving peanut OIT were profoundly desensitized to exposure to peanut protein. As summarized in Table 4, as measured by highest tolerated dose with no more than mild symptoms at exit DBPCFC, 58.3% the OIT patient arm tolerated a 1,000 mg dose (2,043 mg cumulative). In contrast, only 2.3% of the patient arm receiving placebo tolerated the 1,000 mg dose. This profound desensitization was achieved after only approximately 12 weeks of maintenance dosing.

TABLE 4
Desensitization Response Rates

| Exit DBPCFC | AR101 | Placebo |
|---|---|---|
| highest tolerated dose | (N = 132) | (N = 43) |
| None | 0 | 2 |
| 1 mg | 0 | 3 |
| 3 mg | 0 | 5 |
| 10 mg | 1 | 10 |
| 30 mg | 2 | 7 |
| 100 mg | 6 | 6 |
| 300 mg | 7 | 3 |
| 600 mg | 13 | 3 |
| 1,000 mg | 77 | 1 |
| Subjects who did not | 26 | 3 |
| have exit DBPCFC | | |

Anaphylactic reactions: No deaths or suspected, unexpected serious adverse reactions (SUSARs) were observed. Mild and/or moderate anaphylactic reactions were reported in all study periods (initial escalation, up-dosing, and maintenance) for the peanut protein OIT arm, as summarized in Table 5. Surprisingly, even though maintenance phase subjects had underwent 20-40 weeks of up-dosing and progressed to a 300 mg/day maintenance dose, the percent of subjects experiencing an anaphylactic reaction during the up-dosing period was nearly identical to the percent of subjects experiencing an anaphylactic reaction during the maintenance period (7.2% of subjects during the up-dosing period compared with 7.4% of subjects during the maintenance period; see Table 5). Although the treatment population had been initially desensitized to a 300 mg/day dose of peanut protein as a result of the up-dosing period, the incidence of systemic allergic reactions, such as mild/moderate anaphylactic reactions, was not changed during the maintenance period.

Treatment-emergent adverse events leading to discontinuation of study product: A summary of treatment-emergent adverse-events leading to discontinuation of study product is below in Table 6. These adverse events resulted in 3.8% of the OIT patient arm discontinuing during the initial escalation portion of the treatment period and 5.6% of the OIT patient arm discontinuing during the up-dosing portion of the treatment period. Surprisingly, during the maintenance period, zero subjects in the peanut OIT arm experienced a treatment-emergent adverse event leading to discontinuation. During the initial escalation and up-dosing portions of the treatment period, gastrointestinal disorders were the leading cause of discontinuation of study product. Further, as discussed in the preceding section, the OIT study population continued to experience mild/moderate anaphylactic reactions during the maintenance period, whereas, unexpectedly, discontinuations due to gastrointestinal disorders ceased in the maintenance portion of the treatment period.

TABLE 5
Anaphylactic Episodes by Study Period

| | Initial Escalation | | Up-dosing | | Maintenance | | Overall | |
|---|---|---|---|---|---|---|---|---|
| | AR101 | Placebo | AR101 | Placebo | AR101 | Placebo | AR101 | Placebo |
| | (N = 132) | (N = 43) | (N = 125) | (N = 43) | (N = 108) | (N = 41 | (N = 132) | (N = 43) |
| Number of | 1 | 0 | 11 | 0 | 10 | 2 | 22 | 2 |
| anaphylactic | | | | | | | | |
| reactions | | | | | | | | |

Number of Anaphylactic reactions by trigger

| | | | | | | | | |
|---|---|---|---|---|---|---|---|---|
| Study product | 1 | 0 | 9 | 0 | 9 | 0 | 19 | 0 |
| Peanut food | 0 | 0 | 1 | 0 | 1 | 0 | 2 | 0 |
| Other food | 0 | 0 | 1 | 0 | 0 | 1 | 1 | 1 |
| allergen | | | | | | | | |
| Medication | 0 | 0 | 0 | 0 | 0 | 0 | 0 | 0 |
| Insect sting | 0 | 0 | 0 | 0 | 0 | 0 | 0 | 0 |
| Environmental | 0 | 0 | 0 | 0 | 0 | 0 | 0 | 0 |
| allergen | | | | | | | | |
| Other | 0 | 0 | 0 | 0 | 0 | 1 | 0 | 1 |

Number of subjects experiencing an anaphylactic reaction

| | | | | | | | | |
|---|---|---|---|---|---|---|---|---|
| 1 episode | 1 | 0 | 7 | 0 | 6 | 0 | 11 | 0 |
| 2 episodes | 0 | 0 | 2 | 0 | 2 | 1 | 4 | 1 |
| 3 episodes | 0 | 0 | 0 | 0 | 0 | 0 | 1 | 0 |
| >3 episodes | 0 | 0 | 0 | 0 | 0 | 0 | 0 | 0 |

Number of subjects experiencing an anaphylactic
reaction by maximum severity (Muraro Grading Criteria)

| | | | | | | | | |
|---|---|---|---|---|---|---|---|---|
| Mild | 1 | 0 | 4 | 0 | 5 | 1 | 8 | 1 |
| Moderate | 0 | 0 | 5 | 0 | 3 | 0 | 8 | 0 |
| Severe | 0 | 0 | 0 | 0 | 0 | 0 | 0 | 0 |

TABLE 6
Treatment-emergent Adverse Events Leading to Discontinuation of Study Product

| | Initial Escalation | | Up-Dosing | | Maintenance | | Overall | |
|---|---|---|---|---|---|---|---|---|
| | AR101 | Placebo | AR101 | Placebo | AR101 | Placebo | AR101 | Placebo |
| System Organ Class | (N = 132) | (N = 43) | (N = 125) | (N = 43) | (N = 108) | (N = 41) | (N = 132) | (N = 43) |
| Subjects at least 1 | 5 | 0 | 7 | 1 | 0 | 0 | 12 | 1 |
| adverse event | | | | | | | | |
| Gastrointestinal | 4 | 0 | 5 | 0 | 0 | 0 | 9 | 0 |
| disorders | | | | | | | | |
| Abdominal pain | 3 | 0 | 3 | 0 | 0 | 0 | 6 | 0 |
| Vomiting | 3 | 0 | 3 | 0 | 0 | 0 | 6 | 0 |
| Nausea | 2 | 0 | 3 | 0 | 0 | 0 | 5 | 0 |
| Oral pruritus | 0 | 0 | 1 | 0 | 0 | 0 | 1 | 0 |
| Respiratory/thoracic/ | 4 | 0 | 1 | 0 | 0 | 0 | 5 | 0 |
| mediastinal disorders | | | | | | | | |
| Cough | 1 | 0 | 0 | 0 | 0 | 0 | 1 | 0 |
| Dyspnoea | 1 | 0 | 0 | 0 | 0 | 0 | 1 | 0 |
| Nasal congestion | 1 | 0 | 0 | 0 | 0 | 0 | 1 | 0 |
| Oropharyngeal pain | 0 | 0 | 1 | 0 | 0 | 0 | 1 | 0 |
| Rhinorrhoea | 1 | 0 | 0 | 0 | 0 | 0 | 1 | 0 |
| Skin and subcutaneous | 1 | 0 | 1 | 0 | 0 | 0 | 2 | 0 |
| tissue disorders | | | | | | | | |
| Erythema | 1 | 0 | 0 | 0 | 0 | 0 | 1 | 0 |
| Urticaria chronic | 0 | 0 | 1 | 0 | 0 | 0 | 1 | 0 |
| General disorders and | 1 | 0 | 0 | 1 | 0 | 0 | 1 | 1 |
| administration site | | | | | | | | |
| conditions | | | | | | | | |
| Malaise | 1 | 0 | 0 | 0 | 0 | 0 | 1 | 0 |
| Granuloma | 0 | 0 | 0 | 1 | 0 | 0 | 0 | 1 |
| Anaphylactic reaction | 0 | 0 | 1 | 0 | 0 | 0 | 1 | 0 |
| Infections and | 0 | 0 | 1 | 0 | 0 | 0 | 1 | 0 |
| infestations | | | | | | | | |
| Vascular disorders | 0 | 0 | 1 | 0 | 0 | 0 | 1 | 0 |

### Example 2

### Clinical Safety Trials of AR101

Further to the study discussed in Example 1, the efficacy and safety of the peanut protein formulation (AR101) in a characterized desensitization oral immunotherapy regimen in peanut-allergic individuals was also studied in the following four clinical trials. Two studies, designated ARC003 and ARC007, are completed, and their open-label extension studies, designated ARC004 and ARC011, are ongoing. For the ongoing studies, the data presented are from a cutoff date of Dec. 15, 2018.

ARC003, also designated PALISADE, was a large, double-blind, placebo-controlled Phase 3 study of AR101 in patients aged 4 to 55 years with peanut allergy, and was the first trial to include double-blind, placebo-controlled food challenges (DBPCFCs) at both entry and exit. See Jones et al., “Efficacy and Safety of AR101 in Peanut Allergy: Results from a Phase 3, Randomized, Double-Blind, Placebo-Controlled Trial (PALISADE),” J. Allergy Clin. Immunol. 141(2), suppl. AB400 (2018); and Vickery et al., “AR101 Oral Immunotherapy for Peanut Allergy,” N. Engl. J. Med., vol 379, no. 21, pp. 1991-2001 (2018). The primary study population comprised 499 patients aged 4 to 17 years (374 AR101, 125 placebo). Subjects were randomized 3:1 into treatment or placebo, received an initial dose escalation over two days from 0.5 to 6 mg peanut protein, completed a ˜20-40 week up-dosing phase from 3 mg to 600 mg peanut protein, and entered a ˜24-28 week maintenance phase with daily 300 mg peanut protein.

ARC004 is an ongoing open-label safety study for ARC003 completers. Group 1 of ARC004 were former placebo-treated patients from ARC003. Group 1 patients underwent a similar initial dose escalation and up-dosing phase as AR101-treated individuals in ARC003. Group 1 patients then underwent a ˜24 week maintenance phase with 300 mg per day of peanut protein. After the ˜24 weeks of maintenance, Group 1 patients entered a target of 88-136 weeks of extended maintenance with daily, biweekly (twice a week), weekly, or QOW (every other week), dosing as tolerated. Group 2 of ARC004 were former AR101-treated patients from ARC003. Group 2 patients entered an extended maintenance phase targeting ˜28-84 weeks of variable duration and dosing regimens across 3 cohorts. The study included 380 AR101-treated patients.

ARC007, also designated RAMSES, was a large, double-blind, randomized, placebo-controlled safety study designed to provide a larger patient data set for assessment of AR101 safety. The study included initial dose escalation and up-dosing phases, but no maintenance phase was included. No screening DBPCFC was performed, consistent with the usual clinical diagnosis of peanut allergy. Patients in ARC007 underwent an initial dose escalation over 2 days from 0.5 to 6 mg peanut protein followed by an up-dosing phase of ˜20-48 weeks from 3 mg to 300 mg daily peanut protein. The study includes at least 337 AR101-treated individuals and 168 placebo patients.

ARC011 is an ongoing, open-label, long-term safety study which included patients from ARC007. AR101-treated patients from ARC007 enter a target ˜24-28 week maintenance phase of 300 mg peanut protein daily. The study includes 226 AR101-treated individuals.

The safety data were analyzed together in two overlapping populations among the four clinical trials (collectively, the “Safety Population”). The Safety Population included the “Controlled Population” and the “Integrated Population.” The Controlled Population included 709 AR101-treated patients and 292 placebo patients during initial escalation and up-dosing phase combined in placebo-controlled studies (ARC003 and ARC007), and 310 AR101-treated patients and 118 placebo-treated patients during 300 mg daily maintenance treatment in ARC003 alone. The Integrated Population (ARC003, ARC007, ARC004, and ARC011) included 812 AR101-treated patients during initial escalation, 794 patients during the up-dosing phase, and 662 patients during 300 mg peanut protein daily maintenance therapy.

Because allergic adverse events were anticipated with the administration of AR101, safety information was collected in a comprehensive manner. Adverse event (AE) information was collected in study diaries that patients completed after study product dosing at home and by clinicians at study sites for dosing under medical supervision. Patients were prohibited from using antihistamines to ensure mild or moderate symptoms of AR101 were not masked during study treatment. For this analysis, the term “anaphylaxis” was used to distinguish a subset of anaphylactic reaction events that were severe, life-threatening, or fatal.

The adverse events of the controlled population over both the initial dose escalation (IDE) and the up-dosing phase are summarized below in Table 7.

TABLE 7
Summary of IDE and Up-Dosing Adverse
Events (Controlled Population)

| | ARC003 and ARC 007 IDE and Up-Dosing | | | |
|---|---|---|---|---|
| | AR101 | | Placebo | |
| | (N = 709) | | (N = 292) | |
| Total no. exposure-years | 294.35 | | 120.23 | |
| Total no. adverse events | 22934 | (77.9) | 3208 | (26.7) |
| (exposure-adjusted) | | | | |
| No. (%) patients with at | | | | |
| least one: | | | | |
| Adverse event | 694 | (97.9%) | 269 | (92.1%) |
| By maximum severity | | | | |
| Grade 1: Mild | 325 | (45.8%) | 180 | (61.6%) |
| Grade 2: Moderate | 348 | (49.1%) | 84 | (28.8%) |
| Grade 3: Severe | 20 | (2.8%) | 4 | (1.4%) |
| Grade 4: Life-threatening | 1 | (0.1%) | 0 | |
| Grade 5: Death | 0 | | 1 | (0.3%) |
| Led to study product | 80 | (11.3%) | 7 | (2.4%) |
| discontinuation | | | | |
| Systemic allergic reaction | 67 | (11.3%) | 11 | (3.8%) |
| Associated with non-study | 81 | (11.4%) | 56 | (19.2%) |
| product food allergen | | | | |
| exposure | | | | |
| Serious adverse event | 6 | (0.8%) | 2 | (0.7%) |

The adverse events of the controlled population over 300 mg daily maintenance are summarized below in Table 8.

TABLE 8
Summary of 300 mg Maintenance Adverse
Events (Controlled Population)

| ARC003 300 mg/day maintenance | | |
|---|---|---|
| | AR101 | Placebo |
| | (N = 310) | (N = 118) |

| Total no. exposure-years | 149.54 | | 57.55 | |
|---|---|---|---|---|
| Total no. adverse events | 4041 | (27.0) | 650 | (11.3) |
| (exposure-adjusted) | | | | |
| No. (%) patients with at | | | | |
| least one: | | | | |
| Adverse event | 270 | (87.1%) | 94 | (79.7%) |
| By maximum severity | | | | |
| Grade 1: Mild | 161 | (51.9%) | 57 | (48.3%) |
| Grade 2: Moderate | 101 | (32.6%) | 37 | (31.4%) |
| Grade 3: Severe | 8 | (2.6%) | 0 | |
| Grade 4: Life-threatening | 0 | | 0 | |
| Grade 5: Death | 0 | | 0 | |
| Led to study product | 4 | (1.3%) | 0 | |
| discontinuation | | | | |
| Systemic allergic reaction | 27 | (8.7%) | 2 | (1.7%) |
| Associated with non-study | 28 | (9.0%) | 24 | (20.3%) |
| product food allergen | | | | |
| exposure | | | | |
| Serious adverse event | 4 | (1.3%) | 1 | (0.8%) |

As expected with desensitization treatment, the proportion of patients with at least 1 adverse event of any severity or relationship to study product was higher during initial dose escalation and up-dosing combined (97.9% AR101, 92.1% placebo) than during 300 mg/day dosing (87.1% AR101, 79.7% placebo). In the Controlled Population, adverse events led to discontinuation of study treatment in 11.3% of patients in the AR101 group and 2.4% in the placebo group during initial dose escalation and up-dosing combined, compared with 1.3% of patients in the AR101 group and no patient in the placebo group during the 300 mg peanut protein daily maintenance dosing.

The most common adverse events that led to discontinuation of study treatment were gastrointestinal-related responses (abdominal pain, vomiting, and nausea) that occurred during initial dose escalation and up-dosing, as summarized in Table 9, below. Systemic allergic reactions led to discontinuation of study treatment in 11 patients (1 event of anaphylaxis) during initial dose escalation (IDE) and up-dosing combined, and 2 patients (1 event of anaphylaxis) during 300 mg peanut protein daily maintenance dosing.

TABLE 9
Adverse Events Leading to Discontinuation of Study Treatment
in >1 Patient by Preferred Term (Controlled Population)

| | IDE and Up-Dosing | | | 300 mg Maintenance | |
|---|---|---|---|---|---|
| Category | AR101 | | Placebo | AR101 | Placebo |
| Preferred Term | (N = 709) | | (N = 292) | (N = 310) | (N= 118) |
| Patients with at least 1 | 80 | (11.3%) | 7 (2.4%) | 4 (1.3%) | 0 |
| adverse event | | | | | |
| Abdominal pain | 25 | (3.5%) | 1 (0.3%) | 0 | 0 |
| Vomiting | 19 | (2.7%) | 0 | 0 | 0 |
| Nausea | 13 | (1.8%) | 0 | 0 | 0 |
| Anaphylactic reaction | 11 | (1.6%) | 0 | 2 (0.6%) | 0 |
| Abdominal | 7 | (1.0%) | 1 (0.3%) | 0 | 0 |
| discomfort | | | | | |
| Urticaria | 6 | (0.8%) | 2 (0.7%) | 0 | 0 |
| Throat irritation | 6 | (0.8%) | 1 (0.3%) | 0 | 0 |
| Abdominal pain | 6 | (0.8%) | 0 | 0 | 0 |
| upper | | | | | |
| Throat tightness | 5 | (0.7%) | 2 (0.7%) | 0 | 0 |
| Wheezing | 4 | (0.6%) | 1 (0.3%) | 1 (0.3%) | 0 |
| Cough | 4 | (0.6%) | 0 | 0 | 0 |
| Oral pruritus | 4 | (0.6%) | 0 | 0 | 0 |
| Pruritus | 3 | (0.4%) | 1 (0.3%) | 0 | 0 |
| Eosinophilic | 3 | (0.4%) | 0 | 0 | 0 |
| esophagitis | | | | | |
| Gastroesophageal | 3 | (0.4%) | 0 | 0 | 0 |
| reflux disease | | | | | |
| Retching | 3 | (0.4%) | 0 | 0 | 0 |
| Rhinorrhoea | 2 | (0.3%) | 1 (0.3%) | 0 | 0 |
| Lip swelling | 2 | (0.3%) | 0 | 0 | 0 |
| Salivary | 2 | (0.3%) | 0 | 0 | 0 |
| hypersecretion | | | | | |
| Rash | 2 | (0.3%) | 0 | 0 | 0 |
| Chest pain | 2 | (0.3%) | 0 | 0 | 0 |
| Chest discomfort | 1 | (0.1%) | 0 | 1 (0.3%) | 0 |
| Anxiety | 1 | (0.1%) | 1 (0.3%) | 0 | 0 |

Key adverse events of clinical interest were pre-specified for analysis and included systemic allergic reactions (including anaphylaxis), use of epinephrine as rescue medication, and chronic/recurrent GI adverse events leading to study discontinuation, including eosinophilic esophagitis (EoE). A summary of adverse events of clinical interest for the Controlled Population is summarized in Table 10, below. The proportions of patients who experienced adverse events of clinical interest were higher in patients treated with AR101 compared with placebo.

TABLE 10
Adverse Events of Clinical Interest (Controlled Population)

| | Patients with ≥ 1 Adverse Event of Clinical Interest | | | |
|---|---|---|---|---|
| | IDE and Up-Dosing | | 300 mg Maintenance | |
| | AR101 | Placebo | AR101 | Placebo |
| | (N = 709) | (N = 292) | (N = 310) | (N = 118) |
| Systemic allergic | 67 (9.4%) | 11 (3.8%) | 27 (8.7%) | 2 (1.7%) |
| reactions | | | | |
| Anaphylaxis | 4 (0.6%) | 0 | 1 (0.3%) | 0 |
| Use of epinephrine | 74 (10.4%) | 14 (4.8%) | 24 (7.7%) | 4 (3.4%) |
| Chronic/recurrent | 36 (5.1%) | 0 | 0 | 0 |
| gastrointestinal | | | | |
| adverse events | | | | |
| leading to study | | | | |
| discontinuation | | | | |
| Eosinophilic | 3 (0.4%) | 0 | 0 | 0 |
| esophagitis | | | | |

In the Controlled Population, 81 systemic allergic reactions were reported in 67 patients (9.4%) in the AR101 group and 11 were reported in 11 patients (3.8%) in the placebo group during initial dose escalation and up-dosing combined, and 33 systemic allergic reactions were reported in 27 patients (8.7%) in the AR101 group and 2 were reported in 2 patients (1.7%) in the placebo group during the 300 mg daily maintenance dosing. After adjusting for exposure, the frequency of systemic allergic reactions was 0.28 events per patient-year in the AR101 treatment group during initial dose escalation and up-dosing combined and 0.22 events per patient-year during 300 mg daily maintenance dosing.

In the Integrated Population of 812 patients, 186 events of systemic allergic reaction were reported in 127 patients. Most systemic allergic reactions were mild or moderate in severity. Systemic allergic reactions are summarized for the Integrated Population in Table 11, below. The analysis of systemic allergic reaction events does not include those events occurring during the DBPCFCs of any trial.

TABLE 11
Summary of Systemic Allergic Reaction
Events (Integrated Population)

| | | | 300 mg | |
|---|---|---|---|---|
| | IDE | Up-dosing | maintenance | Overall |
| | (N = 812) | (N = 794) | (N = 662) | (N = 812) |
| Systemic allergic | 5 | 85 | 96 | 186 |
| reactions | | | | |
| Patients with a | | | | |
| systemic allergic | | | | |
| reaction | | | | |
| 1 Episode | 5 (0.6%) | 57 (7.2%) | 55 (8.3%) | 89 (11.0%) |
| 2 Episodes | 0 | 14 (1.8%) | 4 (0.6%) | 25 (3.1%) |
| 3 Episodes | 0 | 0 | 7 (1.1%) | 10 (1.2%) |
| >3 Episodes | 0 | 0 | 2 (0.3%) | 3 (0.4%) |
| Patients with | | | | |
| systemic allergic | | | | |
| reaction by | | | | |
| maximum severity | | | | |
| Mild | 5 (0.6%) | 28 (3.5%) | 28 (4.2%) | 52 (6.4%) |
| Moderate | 0 | 39 (4.9%) | 34 (5.1%) | 65 (8.0%) |
| Severe | 0 | 4 (0.5%) | 6 (0.9%) | 10 (1.2%) |
| (anaphylaxis) | | | | |
| Patients with a | 0 | 2 (0.3%) | 2 (0.3%) | 4 (0.5%) |
| systemic allergic | | | | |
| reaction that was | | | | |
| a serious adverse | | | | |
| event | | | | |
| Patients with a | 3 (0.4%) | 47 (5.9%) | 50 (7.6%) | 89 (11.0%) |
| systemic allergic | | | | |
| reaction that | | | | |
| required | | | | |
| epinephrine | | | | |
| Epinephrine use | 0 | 38 (4.8%) | 47 (7.1%) | 79 (9.4%) |
| other than at | | | | |
| study site | | | | |
| Epinephrine use | 3 (0.4%) | 13 (1.6%) | 4 (0.6%) | 20 (2.5%) |
| at study site | | | | |

When adjusted for exposure, the event rate of systemic allergic reaction decreased after the initial dose escalation phase and then remained relatively consistent over the first year of treatment, as summarized in Table 12, below (PYE, patient-years exposure; IDE, initial dose escalation). The event rate for systemic allergic reactions was 1.14 events per patient-year during initial dose escalation, 0.25 during up-dosing, and 0.20 during 300 mg daily maintenance dosing. Similarly, epinephrine use associated with systemic allergic reaction decreased after initial dose escalation and then remained relatively consistent.

TABLE 12
Exposure-Adjusted Rates for Systemic Allergic
Reactions (Integrated Population)

| | | | 300 mg |
|---|---|---|---|
| | IDE | Up-Dosing | Maintenance |
| | (N = 812) | (N = 794) | (N = 662) |
| | PYE = 4.40 | PYE = 335.04 | PYE = 479.19 |
| Number of systemic | 5 (1.14) | 85 (0.25) | 96 (0.20) |
| allergic reactions | | | |
| Mild | 5 (1.14) | 36 (0.11) | 45 (0.09) |
| Moderate | 0 | 45 (0.13) | 45 (0.09) |
| Severe | 0 | 4 (0.01) | 6 (0.01) |
| Administered | 3 (0.68) | 55 (0.16) | 66 (0.14) |
| epinephrine | | | |

Systemic allergic reactions are expected to decrease in frequency with longer duration of treatment due to increasing desensitization and continued immunomodulation.

In the integrated population, anaphylaxis was reported in 10 patients: 4 patients during up-dosing and 6 patients during 300 mg daily maintenance dosing (1 at 14-26 weeks and 5 at 27-52 weeks), as summarized in Table 13, below.

TABLE 13
Summary of Anaphylaxis Events (Integrated Population)

| | | | | | Action |
|---|---|---|---|---|---|
| | AR101 | | | Epinephrine | Taken with |
| Age/Sex | Dose | Period | Serious | Use | AR101 |
| 10/M | 40 mg | Up-dosing | No | Yes | Reduced |
| 14/F | 120 mg | Up-dosing | No | No | Interrupted |
| 14/F | 80 mg | Up-dosing | No | Yes | Interrupted |
| 17/M | 6 mg | Up-dosing | No | Yes | Discontinued |
| 5/F | 240 mg | Maintenance | No | No | Interrupted |
| 15/F | 300 mg | Maintenance | Yes | Yes | Discontinued |
| 4/F | 300 mg | Maintenance | No | Yes | Interrupted |
| 14/F | 300 mg | Maintenance | No | Yes | Interrupted |
| 13/F | 300 mg | Maintenance | No | Yes | Discontinued |
| 5/F | 300 mg | Maintenance | No | Yes | Interrupted |

Efforts were taken to determine associated extrinsic factors (cofactors) to further understand systemic allergic reactions reported during AR101 clinical studies. The most complete and reliable data were available for the completed studies ARC003 and ARC007, which make up the Controlled Population. There were 117 systemic allergic reactions in 89 patients aged 4 to 17 years in the ARC003 and ARC007 AR101 group. Systemic allergic reactions, including anaphylaxis, were assessed for contributing cofactors by reviewing the patient narratives submitted in the final clinical study reports for studies ARC003 and ARC007.

In patients aged 4 to 17 years, overall, 117 systemic allergic reactions of any severity or trigger (study product, food allergen, or other allergen) were reported in 89 patients (12.6%) in the AR101 group and 13 were reported in 13 patients (4.5%) in the placebo group. Of these, a total of 57 AR101-treated patients (64.0%) and 3 placebo-treated patients (23.1%) had 1 or more contributing cofactors. Cofactors contributing to systemic allergic reaction in at least 10% of patients in either treatment group overall were exercise (40.4% AR101, 7.7% placebo), exposure to hot water (13.5% AR101, 0% placebo), intercurrent illness (12.4% AR101, 0% placebo), fasting (11.2% AR101, 0% placebo), and other reasons such as related to the food matrix used to prepare the study product, stress, etc. (14.6% AR101, 7.7% placebo). Additional contributing factors reported in 2.2% to 5.6% of AR101-treated patients overall included menstruation, sleep deprivation, NSAID use, and uncontrolled asthma.

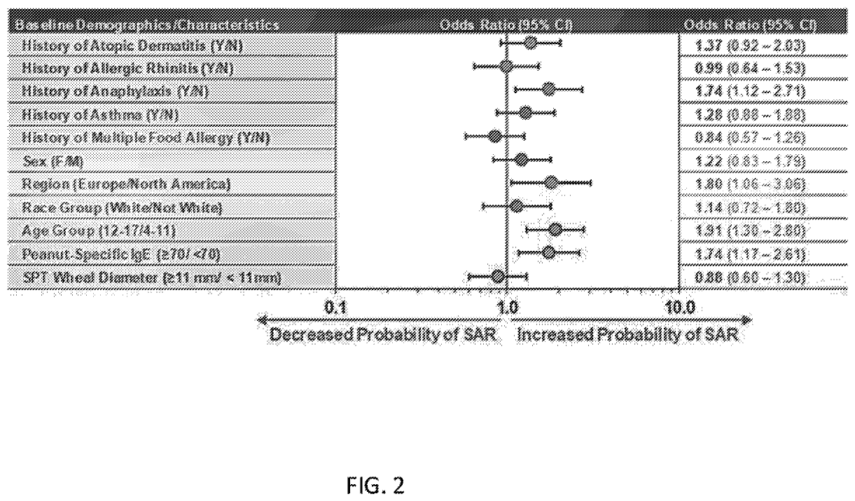

Some baseline factors were associated with increased odds of systemic allergic reaction (odds ratio of 1.74-1.91 for the Integrated Population), including a history of anaphylaxis, Europe region, older pediatric age group (12-17 years), and baseline peanut specific IgE≥70 kU_{A}/L. An analysis of the probability of any systemic allergic reaction by baseline characteristics in the Integrated Population is presented in FIG. 2. History of anaphylaxis (Y/N, odds ratio 1.74; 95% CI:1.12-2.71), region (Europe/North America, odds ratio 1.80; 95% CI:1.06-3.06), age group (12-17/4-11, odds ratio 1.91; 95% CI: 1.30-2.80), and peanut-specific IgE (≥70/<70 kU_{A}/L, odds ratio 1.74, 95% CI:1.17-2.61) had 95% Cis that did not encompass (were above) odds ratio of 1.

In the Controlled Population, demographics and baseline clinical characteristics associated with systemic allergic reactions were analyzed from an earlier set of patient results. At the time of the analysis, 12.4% of AR101-treated subjects (88/709) vs 4.5% placebo-treated subjects (13/292) reported a systemic allergic reaction of any severity. Univariate and multivariate logistic regression analyses were run, controlling for treatment group (AR101, placebo) to assess risk factors for systemic allergic reactions of any severity, including the following baseline variables: sex; age; race; region (Europe/North America); history of peanut-related anaphylaxis, atopic dermatitis, allergic rhinitis, asthma, and multiple food allergies; peanut-specific IgE (psIgE); skin prick test (SPT) wheal diameter. For each variable, subjects were stratified and the odds ratio (OR; 95% confidence interval) was calculated. Factors that increased likelihood of experienced a systemic allergic reaction include history of anaphylaxis (yes/no; 2.18, 95% confidence interval (CI):1.31-3.63); European region (Europe/North America; 2.12, 95% CI:1.19-3.77); age group (12-17-years-old/4-11-years-old; 2.08, 95% CI:1.37-3.16); and baseline peanut-specific-IgE group (>70/<70 kU_{A}/L; 1.70, 95% CI:1.09-2.63). The 95% CIs for the other variables encompassed OR=1.00, suggesting no difference in risk of systemic allergic reactions. The same results were observed in the multivariate analyses.

The median peanut skin prick test wheal diameter and peanut-specific IgE (psIgE) at entry were used to categorize the AR101-treated 4-17-year-old patients of the ARC003 portion of the Controlled Population into 2 cohorts (above and below median). The proportion of subjects (treatment difference, 95% CI) tolerating 300 mg, 600 mg, or 1000 mg peanut protein at the exit DBPCFC without dose-limiting symptoms were compared based on the median cut-point. In the intention-to-treat population that completed the exit DBPCFC (AR101 n=372, placebo n=124), 67.2% vs 4.0% tolerated 600 mg at exit DBPCFC without dose-limiting symptoms. The ability to tolerate 600 mg peanut protein at exit DBPCFC (AR101 vs placebo) was similar to the overall population, as summarized in Table 14, below, irrespective of the baseline SPT wheal diameter median cut-point (11mm; Q1, Q3: 9.0, 15.0). Similarly, baseline psIgE levels above or below the median (70.0 kU_{A}/L; Q1, Q3: 19.70, 202.00) were not predictive of tolerating 600 mg peanut protein at exit DBPCFC. Further, the ability to tolerate 300 mg or 1000 mg peanut protein at exit DBPCFC was not predicted by baseline SPT wheal diameter or psIgE.

TABLE 14
Proportion of Subjects Tolerating 600 mg of Peanut Protein
at Exit DBPCFC by Peanut Sensitization Status

| | | | | | AR101-Placebo |
|---|---|---|---|---|---|
| | | | AR101 | Placebo | Treatment Difference, |
| | | | % (total n) | % (total n) | % (95% CI) |
| Overall | | | 67.2 (N = 372) | 4.0 (N = 124) | 63.2 (53.0, 73.3) |
| SPT wheal | <11 | mm | 70.3 (N = 172) | 4.1 (N = 49) | 66.3 (50.5, 82.0) |
| diameter | ≥11 | mm | 64.3 (N = 199) | 4.0 (N = 75) | 60.3 (47.1, 73.6) |
| psIgE | <70 | kU_{A}/L | 69.9 (N = 186) | 8.6 (N = 58) | 61.3 (46.6, 75.9) |
| | ≥70 | kU_{A}/L | 64.5 (N = 186) | 0 (N = 65) | 64.5 (50.4, 78.6) |

What is claimed is:

1. A method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising:
obtaining a peanut-specific IgE level of the subject during the maintenance phase; and
assessing the risk of a systemic allergic response for the subject based on the obtained peanut-specific IgE level, wherein a peanut-specific IgE level above a predetermined threshold indicates an increased risk of a systemic allergic response.

2. The method of claim 1, wherein the predetermined threshold is a peanut-specific IgE level of about 70 kU_{A}/L.

3. A method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising:
obtaining a peanut-specific IgE level of the subject during the maintenance phase; and
assessing the risk of a systemic allergic response for the subject based on the obtained peanut-specific IgE level, wherein a peanut-specific IgE level above a baseline peanut-specific IgE level of the subject indicates an increased risk of a systemic allergic response compared to a baseline risk.

4. The method of any one of claims 1-3, wherein the subject tolerates a dose of about 1000 mg or more of peanut protein when the peanut-specific IgE level is obtained and the risk of the systemic allergic response is assessed.

5. The method of any one of claims 1-4, wherein the subject tolerates a cumulative dose of about 2043 mg or more of peanut protein when the peanut-specific IgE level is obtained and the risk of the systemic allergic response is assessed.

6. The method of any one of claims 1-5, further comprising administering to the subject the composition comprising the peanut protein according to the oral immunotherapy schedule.

7. A method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising:
desensitizing the subject to peanut protein using the oral immunotherapy until the subject tolerates a dose of 1000 mg or more peanut protein;
obtaining a peanut-specific IgE level when the subject tolerates the dose of 1000 mg or more peanut protein; and
assessing the risk of a systemic allergic response in the subject based on the obtained peanut-specific IgE level, wherein a peanut-specific IgE level above a predetermined threshold indicates an increased risk of a systemic allergic response.

8. The method of claim 7, wherein the predetermined threshold is a peanut-specific IgE level of about 70 kU_{A}/L.

9. A method of assessing risk of a systemic allergic response in a subject being treated for a peanut allergy by an oral immunotherapy comprising administering to the subject a composition comprising peanut protein according to an oral immunotherapy schedule comprising an up-dosing phase and a maintenance phase, the method comprising:
desensitizing the subject to peanut protein using the oral immunotherapy until the subject tolerates a dose of 1000 mg or more peanut protein;
obtaining a peanut-specific IgE level when the subject tolerates the dose of 1000 mg or more peanut protein; and
assessing the risk of a systemic allergic response in the subject based on the obtained peanut-specific IgE level, wherein a peanut-specific IgE level above a baseline peanut-specific IgE level of the subject indicates an increased risk of a systemic allergic response compared to a baseline risk.

10. The method of any one of claims 1-9, wherein the peanut-specific IgE level is obtained from the subject less than 6 months from the start of the maintenance phase.

11. The method of any one of claims 1-10, wherein the peanut-specific IgE level is obtained from the subject about 3 months to less than 6 months from the start of the maintenance phase.

12. The method of any one of claims 1-11, wherein the peanut-specific IgE level is obtained from the subject about 3 months from the start of the maintenance phase.

13. A method of assessing risk of a systemic allergic response in a subject during the course of treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising:
obtaining a peanut-specific IgE level of the subject prior to the start of the oral immunotherapy; and
assessing the risk of a systemic allergic response for the subject during the course of the oral immunotherapy based on the obtained peanut-specific IgE level, wherein a peanut-specific IgE level above a predetermined threshold indicates an increased risk of a systemic allergic response during the course of oral immunotherapy.

14. The method of claim 13, wherein the predetermined threshold is a peanut-specific IgE level of about 70 kU_{A}/L.

15. The method of claim 13 or 14, further comprising administering to the subject the composition comprising the peanut protein according to the oral immunotherapy schedule.

16. The method of any one of claims 13-15, wherein the risk of systemic allergic response is assessed prior to the start of the oral immunotherapy.

17. The method of any one of claims 1-16, wherein obtaining the peanut-specific IgE level comprises measuring the peanut-specific IgE level.

18. The method of any one of claims 1-17, wherein obtaining the peanut-specific IgE level comprises receiving the peanut-specific IgE level.

19. The method of any one of claims 1-18, wherein the age the subject is obtained, and assessment of the risk of a systemic allergic response is further based on the age of the subject, wherein an age of 12 years of age or older indicates an increased risk of a systemic allergic response compared to an age between about 4 years old and about 11 years old.

20. The method of any one of claims 1-18, wherein the age the subject is obtained, and assessment of the risk of a systemic allergic response is further based on the age of the subject, wherein an age between 12 years of age and 17 years of age indicates an increased risk of a systemic allergic response compared to an age between about 4 years old and about 11 years old.

21. The method of any one of claims 1-18, wherein the sex the subject is obtained, and assessment of the risk of a systemic allergic response is further based on the sex of the subject, wherein the subject being female indicates an increased risk of a systemic allergic response compared to the subject being male.

22. The method of any one of claims 1-18, wherein the sex and age of the subject are obtained, and assessment of the risk of a systemic allergic response is further based on the sex and age of the subject, wherein the subject being female 12 years of age or older indicates an increased risk of a systemic allergic response compared to the subject not being a female 12 year of age or older.

23. The method of any one of claims 1-18, wherein the sex and age of the subject are obtained, and assessment of the risk of a systemic allergic response is further based on the sex and age of the subject, wherein the subject being female between 12 years of age and 17 years of age indicates an increased risk of a systemic allergic response compared to the subject not being a female between 12 year of age and 17 years of age.

24. The method of any one of claims 1-23, wherein the subject is about 4 years old or older.

25. The method of claim 24, wherein the subject is about 4 years old to less than 18 years old.

26. A method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising:
obtaining the age of the subject; and
assessing the risk of a systemic allergic response for the subject during the course of the oral immunotherapy based on the age, wherein an age of 12 years of age or older indicates an increased risk of a systemic allergic response compared to an age between about 4 years old and about 11 years old.

27. The method of claim 26, wherein an age between 12 years of age and 17 years of age indicates an increased risk of a systemic allergic response compared to an age between about 4 years old and about 11 years old.

28. The method of claim 26 or 27, wherein the sex the subject is obtained, and assessment of the risk of a systemic allergic response is further based on the sex of the subject, wherein the subject being female indicates an increased risk of a systemic allergic response compared to the subject being male.

29. A method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising:
obtaining the sex of the subject; and
assessing the risk of a systemic allergic response for the subject during the course of the oral immunotherapy based on the sex, wherein the subject being female indicates an increased risk of a systemic allergic response compared to a male subject.

30. A method of assessing risk of systemic allergic response in a subject during the course of a treatment for a peanut allergy by an oral immunotherapy, wherein the oral immunotherapy comprises administering to the subject a composition comprising peanut protein according to an oral immunotherapy schedule comprising an up-dosing phase and a maintenance phase, the method comprising:
obtaining the age and sex of the subject; and
assessing the risk of a systemic allergic response for the subject based on the age and the sex, wherein the subject being female 12 years of age or older indicates an increased risk of a systemic allergic response compared to the subject not being a female 12 year of age or older.

31. The method of claim 30, wherein the subject being female between 12 years of age and 17 years of age indicates an increased risk of a systemic allergic response compared to the subject not being a female between 12 year of age and 17 years of age.

32. The method of any one of claims 26-31, further comprising obtaining a baseline peanut-specific IgE level, wherein assessment of the risk of a systemic allergic response during the course of the oral immunotherapy is further based on the obtained peanut-specific IgE level, and wherein a peanut-specific IgE level above a predetermined threshold indicates an increased risk of a systemic allergic response during the course of oral immunotherapy.

33. The method of claim 32, wherein the predetermined threshold is a peanut-specific IgE level of about 70 kU_{A}/L.

34. The method of any one of claims 26-33, wherein the risk of a systemic allergic response during the course of the oral immunotherapy is assessed prior to the start of the oral immunotherapy.

35. The method of any one of claims 1-34, wherein the subject undergoes heightened monitoring for a systemic allergic response if the subject is at an increased risk of a systemic allergic response.

36. The method of any one of claims 1-35, further comprising advising the subject to avoid strenuous activity within two hours of administering the composition comprising peanut protein if the subject is determined to be at an increased risk of a systemic allergic response.

37. The method of any one of claims 1-36, further comprising providing a therapeutically effective amount of epinephrine to the subject for administration in response to a systemic allergic response if the subject is determined to be at an increased risk of a systemic allergic response.

38. The method of any one of claims 1-37, wherein the maintenance phase comprises administering to the subject a maintenance phase dose comprising the composition comprising the peanut protein a daily basis.

39. The method of any one of claims 1-38, wherein the maintenance phase comprises administering to the subject a maintenance phase dose comprising about 300 mg or more of the peanut protein.

40. The method of claim 39, wherein the maintenance phase dose is administered to the subject on a daily basis.

41. The method of any one of claims 1-40, wherein the maintenance phase dose comprises about 300 mg of the peanut protein.

42. The method of any one of claims 1-41, wherein the up-dosing phase comprises administering to the subject two or more different doses of the composition comprising the peanut protein comprising between about 0.2 mg allergenic food protein and about the dose of the maintenance phase dose.

43. The method of any one of claims 1-42, wherein the up-dosing phase is between about 20 weeks months and about 44 weeks in length.

44. The method of any one of claims 1-43, wherein the oral immunotherapy further comprises an initial escalation phase.
